(19) United States
(12) Patent Application Publication
(10) Pub. No.: US 2021/0330949 A1
(43) Pub. Date: Oct. 28, 2021

(54) APPLICATOR

(71) Applicant: 3M INNOVATIVE PROPERTIES COMPANY, SAINT PAUL, MN (US)

(72) Inventors: ALAN R. DOMBROWSKI, WOODBURY, MN (US); ROBERT A. ASMUS, HUDSON, WI (US)

(73) Assignee: 3M INNOVATIVE PROPERTIES COMPANY, SAINT PAUL, MN (US)

(21) Appl. No.: 17/267,304

(22) PCT Filed: Aug. 19, 2019
(86) PCT No.: PCT/IB2019/056983

§ 371 Date: Feb. 9, 2021

Related U.S. Application Data
(60) Provisional application No. 62/872,838, filed on Jul. 11, 2019.
(60) Provisional application No. 62/720,682, filed on Aug. 21, 2018.

(51) Int. Cl. A61F 13/40; B65D 1/09; B65D 1/02; B65D 51/16

(52) U.S. Cl. CPC A61M 35/006; B65D 1/095; B65D 51/16; B65D 1/0207; B65D 1/0238

(57) ABSTRACT

An applicator (100) comprising an elongated hollow body (120) comprising a distal portion (123), a proximal portion (124) having an open end, an exterior surface (127), an interior surface (126), and a filter (135). The interior surface of the applicator defines an internal chamber suitable for receiving an ampoule (160) formed of a frangible material located in the internal chamber proximal to the filter. A spacer (185) having a distal end and a proximal end is provided, and a cap (180) having a proximal portion, a distal portion, an exterior surface, and an interior surface is provided that attaches to the open end of the applicator and maintains the ampoule and spacer in their respective desired positions. The applicator comprises a head (140) including an absorbent component (110) that is capable of absorbing the contents of the frangible ampoule and facilitates applying the contents to the desired surface.

### TECHNICAL FIELD

The present disclosure relates to an applicator for applying a liquid to a surface. In particular the present disclosure relates to an applicator including a frangible ampoule.

### BACKGROUND

Barrier products are used to protect the skin of patients who suffer from a variety of conditions including, for example, urinary and fecal incontinence, skin occlusion, ileostomy and colostomy. The presence of high moisture and corrosive enzymes from intestinal fluids particularly can lead to devastating breakdown of the skin, which can lead to fungal infections and denuding and erosion of the skin. Some of the products which can be used to protect the skin from these challenges include cyanoacrylate monomer solutions, solutions that require special storage conditions due to their tendencies to degrade upon exposure to ambient conditions.

One method of maintaining the functionality of solutions including cyanoacrylate monomers is to seal such solutions in a container, such as a glass ampoule that can be crushed to release the solution shortly before use. Applicators for dispensing small aliquots of solution, e.g., 0.5 mL to 2 mL, which are particularly desirable when the area to be covered is less than 30 square inches (200 cm²) typically employ glass ampoules placed into plastic tubes with foam attached to them and do not have levers for crushing the ampoule. To provide for a safe device, a suitably rigid and/or thick plastic wall material must be used to form the plastic tube, but since the tube is generally pinched between the fingers to activate, these devices may be difficult for users to activate, especially with a single hand.

### SUMMARY

In one aspect, provided is an applicator for applying a substance to a surface, the applicator comprising:
a hollow body, wherein the hollow body includes a distal portion, a proximal portion, an exterior surface, an interior surface, an activation region, and a filter;
a frangible ampoule, wherein the frangible ampoule contains the substance;
a cap, wherein the cap includes a proximal portion, a distal portion, an exterior surface, and an interior surface, and;
a spacer, wherein the spacer includes a distal end and a proximal end, wherein the frangible ampoule is located in the hollow body between the filter and the distal end of the spacer, wherein at least a portion of the frangible ampoule is located in the activation region, and wherein the spacer is located between the frangible ampoule and the cap.

Features and advantages of the present disclosure will be further understood upon consideration of the detailed description as well as the appended claims.

### BRIEF DESCRIPTION OF THE DRAWINGS

FIG. 1 shows a perspective view of one embodiment of an applicator of the present disclosure.

FIG. 2 shows an exploded view of the applicator of FIG. 1.

FIG. 3 shows a perspective view of the applicator body and head of the applicator of FIG. 1.

FIG. 4 shows a longitudinal cross-section of the applicator of FIG. 1.

FIG. 5 shows an axial view from the proximal end of the body of the applicator of FIG. 1.

FIG. 6 shows an axial cross-section of the applicator of FIG. 1 through the ampoule in the activation region.

FIG. 6a shows a longitudinal cross-section of another embodiment of the applicator of the present disclosure.

FIG. 7 shows an axial view of the applicator head from the distal end of the applicator of FIG. 1.

FIG. 8 shows a perspective view of the cap and spacer according of the applicator of FIG. 1.

FIG. 8a shows a perspective view of another embodiment of the cap and spacer according of the applicator of FIG. 1

FIG. 9 shows a first cross-sectional view of the cap of FIG. 8 where the channel is not visible.

FIG. 10 shows a second cross-sectional view of the cap of FIG. 8 where the channel is visible.

FIG. 11 shows a perspective view of another embodiment of an applicator cap according to the present disclosure.

FIG. 12 shows a cross-sectional view of another embodiment of an applicator cap and spacer according to the present disclosure.

FIG. 13 shows an exploded view of an applicator including the cap and spacer of FIG. 12.

Repeated use of reference characters in the specification and drawings is intended to represent the same or analogous features or elements of the disclosure. It should be understood that many other modifications and embodiments can be devised by those skilled in the art, which fall within the scope and spirit of the principles of the disclosure. The figures may not be drawn to scale.

### DETAILED DESCRIPTION

### Definitions

The term “a”, “an”, and “the” are used interchangeably with “at least one” to mean one or more of the elements being described.

The term “and/or” means either or both. For example, “A and/or B” means only A, only B, or both A and B.

The terms “including,” “comprising,” and “having,” and variations thereof, are meant to encompass the items listed thereafter, and equivalents thereof, as well as additional items.

The terms “polymer” and “polymeric material” refer to both materials prepared from one monomer such as a homopolymer or to materials prepared from two or more monomers such as a copolymer, terpolymer, or the like. Likewise, the term “polymerize” refers to the process of making a polymeric material that can be a homopolymer, copolymer, terpolymer, or the like. The terms “copolymer” and “copolymeric material” refer to a polymeric material prepared from at least two monomers.

The terms “longitudinal” and “axial” are used to refer to a direction or axis that is generally parallel to a central longitudinal axis of an applicator and generally parallel to the overall direction of substance flow.

The terms “lateral” and “transverse” are used to refer to a direction or axis that is perpendicular to the central longitudinal axis the longitudinal direction.

The terms “vertical” and “normal” are used to refer to a direction or axis that is normal to both the longitudinal and lateral directions (or axes).

The term “proximal” and “distal” are used to represent longitudinal or axial directions, relative to a user (such as, for example, a medical practitioner) using or holding the applicator. That is, the term “distal” is used to refer to the direction away from the medical practitioner (and toward a surface (such as, for example, a skin surface) to be treated, i.e., to which the applicator will apply a substance); and the term “proximal” is used to refer to the direction toward the user (and away from the surface to be treated). For example, the distal end of an applicator is configured to be directed toward, or even pressed against, the surface to be treated, while the proximal end extends away from the surface and toward the user. Similarly, the distal end of any portion or component of an applicator is configured to be directed or oriented toward the surface to be treated and is oriented toward the distal end of the applicator or forms or defines the distal end of the applicator. In addition, the proximal end of any portion or component of an applicator is configured to be directed or oriented away from the surface to be treated and is oriented toward the proximal end of the applicator or forms or defines the proximal end of the applicator.

It is often desirable to apply substances to surfaces. In the medical field, single-use applicators may be used to apply medical products to the skin or other tissues. Medical preparations such as, for example, skin barrier products, antiseptics, adhesion enhancing products, adhesive tape trauma protectants, and pharmaceutical agents such as analgesics may be applied as, for example, liquid solutions containing one or more substances. These solutions may be applied with saturated sponges that are attached to a blade or held with forceps. These sponges are often saturated by soaking them in open pans of solution. Sometimes, sponges with attached handles are provided in a plastic or aluminum foil laminate pouch containing enough liquid to saturate the sponges. In some products the sponges are supplied dry in a sterile “kit” with the antiseptic solutions provided in relatively thin-walled polyethylene bottles. These bottles generally have wall thickness less than about 500 microns. While inexpensive, these techniques are messy and offer little control over inadvertent dripping of the solution into areas where it is undesired.

Alternatively, devices have been developed in an attempt to prevent solution dripping associated with these techniques, and to reduce the time required for application of the antiseptic solution. For example, liquid applicators that hold the liquid in a frangible ampoule and require additional elements to crush the ampoule and release the liquid have been developed. However, existing applicators are often complex to construct, may be difficult or cumbersome to use, and may deliver far more solution than is required for a given treatment, particularly when a relatively small area of tissue requires substance application.

Treatment of small areas of skin with a solution is needed in some situations, such as, for example, in the application of skin barrier products to persons with ostomies, including ileostomies and colostomies. Treatment of these smaller areas may present challenges not encountered when larger treatment areas are involved. For example, because the treatment area is small, an applicator containing a small volume of solution is preferred to minimize wasting what may be a costly solution. Moreover, for conditions such as ostomies, such products may be applied in a nonclinical setting by the person with the condition, creating a need for a simple and easy-to-use applicator that can reliably deliver less than 5 mL (e.g., 0.2-2 mL) of a solution to a surface in need of treatment.

Applicators that can hold a small amount of substance (e.g., less than 5 mL), that allow for sterilization processes, e.g., ethylene oxide exposure, and that may be activated and ergonomically used with one hand are needed for certain applications. The present disclosure provides an applicator that, inter alia, includes a handle large enough to be ergonomic while still providing an ampoule suitable for containing a small amount of a substance, the applicator including a spacer that maintains the ampoule in an activation region of the applicator body so that the ampoule may be fractured while the applicator is held in a single hand.

In one aspect, the present disclosure provides an applicator for applying a substance to a surface. Generally, the applicator comprises an elongated hollow body comprising a distal portion, a proximal portion having an open end, an exterior surface, an interior surface, and a filter. The interior surface of the applicator defines an internal chamber suitable for receiving an ampoule. A first ampoule formed of a frangible material is located in the internal chamber proximal to the filter and contains a substance to be applied. A spacer having a distal end and a proximal end is provided, and a cap having a proximal portion, a distal portion, an exterior surface, and an interior surface is provided that attaches to the open end of the applicator and maintains the ampoule and spacer in their respective desired positions. The applicator includes a head and an elongated hollow body comprising a distal portion, a proximal portion having an open end, an exterior surface, an interior surface, and a filter. The interior surface of the applicator defines an internal chamber suitable for receiving an ampoule. A first ampoule formed of a frangible material may be located in the internal chamber proximal to the filter and contains a substance to be applied. A spacer having a distal end and a proximal end is provided, and a cap having a proximal portion, a distal portion, an exterior surface, and an interior surface is provided that attaches to the open end of the applicator and maintains the ampoule and spacer in their respective desired positions. The applicator further comprises an absorbent component that is capable of absorbing the contents of the frangible ampoule and facilitate applying the contents to the desired surface.

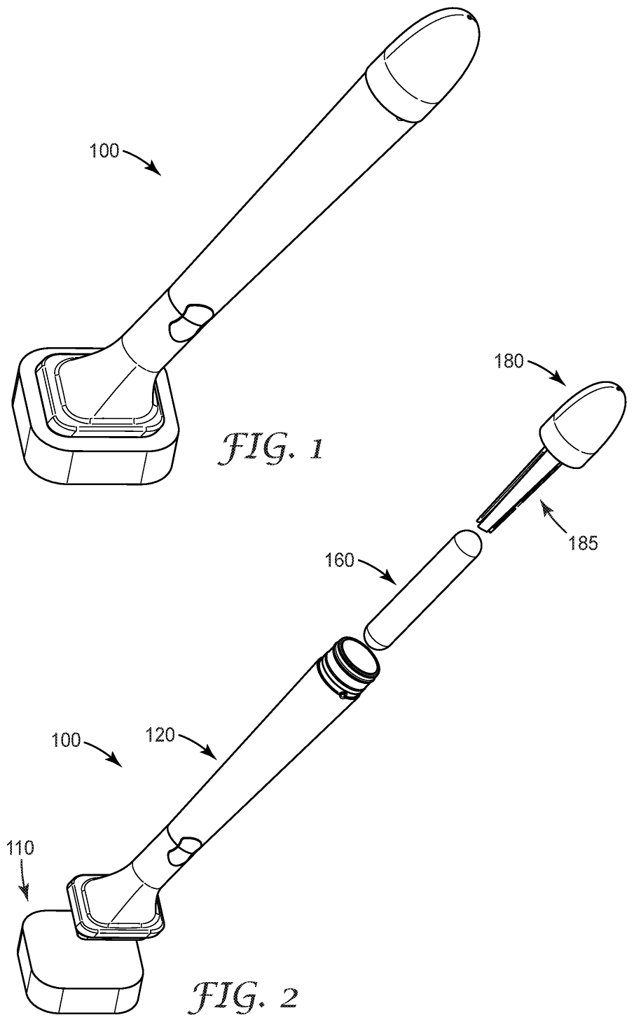

An exemplary applicator 100 according to one embodiment of the present disclosure is illustrated in FIGS. 1-4. The applicator 100 may include an absorbent component 110, a hollow body 120, a frangible ampoule 160, a cap 180, and a spacer 185.

The absorbent component 110 may be formed of any suitable porous substance such as, for example, a sponge, a woven material, a nonwoven material, a screen, a mesh, and combinations thereof. Materials suitable for use as the absorbent component 110 may include, for example, polyester polyurethane and polyester polyether open-cell foams. The absorbent component 110 may be attached to the head 140 at the distal end 123 of the hollow body 120 and may, among other functions, aid in control of the flow rate and distribution of the contents of the frangible ampoule 160 after the contents have been released from the frangible ampoule 160.

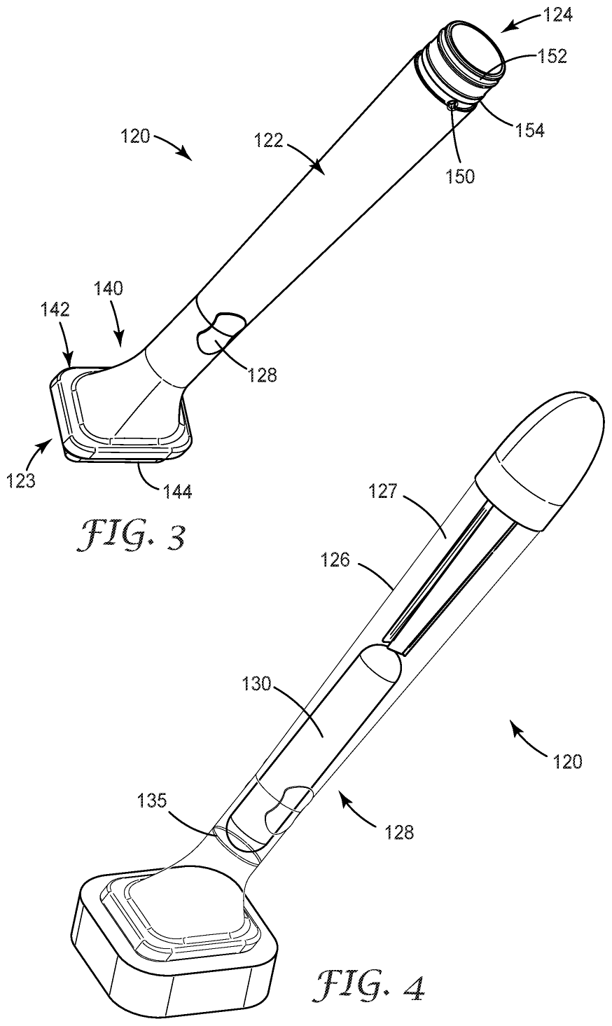

In some embodiments, the absorbent component 110 may be attached to the head 140 at the distal end 123 of the hollow body 120 by, for example, hot plate welding. Referring to FIG. 3, absorbent component 110 may be attached to flange 142 by heating lip 144 to a suitable temperature and then bringing the heated lip 144 and the absorbent component 110 into contact under pressure. In other embodiments, the absorbent component 110 may be attached to hollow body 120 by other means, such as, for example, an adhesive, a spin weld, an ultrasonic weld, or combinations thereof.

The hollow body 120 includes a distal portion 123 and a proximal portion 124, inner surface 126 and outer surface 127. The hollow body 120 includes a handle section 122 that is generally elongated and tapered, e.g., the hollow body 120 may have a frustoconical geometry and may taper as shown along the longitudinal axis of the hollow body 120 from the proximal portion 124 to the distal portion 123 (see FIGS. 1-4 and 13) to facilitate gripping of the applicator 100 by the user and configured to retain the frangible ampoule 160. The handle section 122 further includes activation region 128. Application of a force to the activation region 128 desirably results in the fracture of the frangible ampoule 160 that is contained in the hollow body 120. The force may be applied by, for example, the user holding the applicator along the palm of his/her hand and squeezing the applicator between a finger and thumb of that hand in the activation region 128. The proximal portion 124 may also include vent 150, seal ring 152, and attachment feature 154.

Referring to FIG. 4, frangible ampoule 160 is located within hollow body 120 such that at least a portion of the frangible ampoule 160 is located in the activation region 128 so that when the activation region 128 is compressed the frangible ampoule 160 is fractured. The frangible ampoule 160 may be hermetically sealed and can contain a solution. The frangible ampoule 160 may desirably act as a protective barrier to contact between the solution contained therein and the outer environment. For example, the frangible ampoule 160 may be resistant to environmental changes encountered by the applicator 100 during manufacture, transport, and use of the applicator, such as, for example, the vacuum and chemicals, e.g., ethylene oxide, that the applicator may be exposed to during a sterilization process, conditions that could damage or destroy the solution inside the frangible ampoule 160.

Commonly, the frangible ampoule 160 may be formed of a brittle material, such as, for example, a glass. Suitable brittle materials for forming the frangible ampoule 160 include, for example, a soda-lime glass, a borosilicate glass, an onion skin borosilicate glass, a polymer, a ceramic, and combinations thereof. Such materials are desirably brittle and will fracture when compressed. This is in contrast to relatively flexible materials that would deform when compressed but which must be punctured to release the substance inside. Typically, the substance contained within the frangible ampoule 160 is released by applying external force to the applicator 100 in the activation region 128 sufficient to shatter the frangible ampoule 160. In some embodiments, score lines or other features that provide local areas of weakness in the brittle material may be included to control breaking and/or reduce the force required to break the frangible ampoule 160.

The size and shape of the frangible ampoule 160 is selected to be compatible with the dimensions of the hollow body 120 and the desired volume of substance to be applied. For example, for use in preparation for a small surgical procedure, the amount of substance in the ampoule 160 should generally be sufficient to cover an area of, such as, for example, about 10 square centimeters. For larger surgical procedures, the amount of substance in the ampoule 160 may need to be sufficient to cover at least the torso of a large person, such as, for example, at least about 500-600 square centimeters. The size of the frangible ampoule 160 may be selected to be appropriate for the volume of substance to be delivered in order to minimize product costs and waste. In addition, smaller frangible ampoules 160 may result in products with longer shelf lives. As a nonlimiting example, it has been found that solutions containing cyanoacrylate adhesives have longer shelf lives in small vials compared to larger vials even when the vials are flushed with an inert gas before sealing. Without being bound by theory, it is believed that the smaller interior surface area of a smaller frangible ampoule has fewer sites on the interior surface of the glass that can initiate polymerization of the cyanoacrylate, thereby extending the shelf life of the product.

In some embodiments, the frangible ampoule 160 is selected to apply a substance to a surface having an area such as, for example, about 5 cm² to about 200 square cm². In some embodiments, use of a smaller frangible ampoule 160 permits a frangible ampoule 160 having a thinner wall to be used, thus increasing the ease with which a frangible ampoule 160 may be fractured. In some embodiments, the frangible ampoule 160 is configured to contain, for example, from about 0.2 mL to about 2 mL (e.g., 1 mL) of a substance.

The frangible ampoule 160 contains the substance to be dispensed. Generally, a wide variety of substances can be contained within the frangible ampoule 160, with the selection of the substance influencing the selection of the materials used to construct the frangible ampoule and other parts of the applicator 100, as would be readily understood by one of ordinary skill in the art. Substances contained in the frangible ampoule may include, for example, liquids, gels, suspensions, and pastes. In some embodiments, the substance contained in the ampoule 160 is a liquid composition that includes a volatile carrier and an active agent, such as, for example, an antiseptic agent or a pharmaceutical agent. In some embodiments, the applicator 100 may be particularly useful in dispensing substances having viscosities at room temperature of less than about 10,000 cps. In other embodiments, the applicator may be particularly useful in dispensing substances having viscosities less than about 500 cps.

In some embodiments, the frangible ampoule 160 may contain an antiseptic preparation. Examples of suitable antiseptic preparations include those described in U.S. Pat. No. 4,584,192 and those described in U.S. Pat. No. 4,542,012. Other useful fluids include antiseptic preparations, such as, for example, iodophoric skin tinctures, such as DURAPREP Surgical Solution, commercially available from 3M Company, Saint Paul, Minn., USA. In some embodiments, the ampoule 160 may be filled with a composition that includes an antimicrobial agent such as iodine, an iodine complex (such as, for example, iodophors), chlorhexidine, chlorhexidine salts (such as, for example, chlorhexidine digluconate and chlorhexidine diacetate), or combinations thereof. Other exemplary antimicrobial agents include C2-C5 lower alkyl alcohols, fatty acid monoesters of glycerin and propylene glycol, polymers that include a (C12-C22) hydrophobe and a quaternary ammonium group, polyquaternary amines (such as, for example, polyhexamethylene biguanide), quaternary ammonium silanes, silver, silver salts (such as silver chloride), silver oxide and silver sulfadiazine, methyl, ethyl, propyl and butyl parabens, octenidine, peroxides (such as, for example, hydrogen peroxide and benzoyl peroxide), and the like, as well as combinations thereof. In other embodiments, the frangible ampoule may contain, for example, medical preparations such as skin barrier products, adhesion enhancing products, adhesive tape trauma protectants, and pharmaceutical agents such as analgesics. As a nonlimiting example, the frangible ampoule may contain a skin barrier product, such as CAVILON Advanced Skin Protectant, commercially available from 3M Company, Saint Paul, Minn., USA. In some embodiments, the contents of the frangible ampoule 160 include a cyanoacrylate monomer.

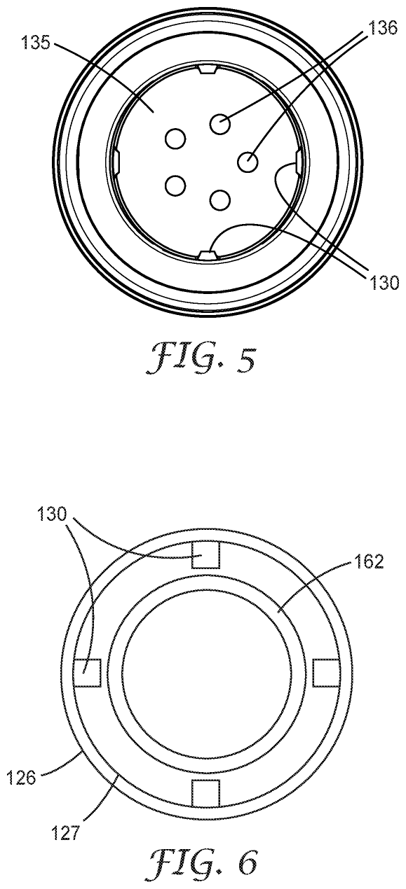

Referring to FIG. 5, the handle 122 of the body 120 includes filter 135, located between the frangible ampoule 160 and the absorbent component 110. As shown, the filter 135 may be integrally molded into the handle 122 and is provided with holes 136. Five holes 136 are shown in FIG. 5, though fewer holes 136, e.g., 3, or more holes 136, e.g., 7, are contemplated. The holes 136 are shown having a circular geometry and uniform size, though other geometries, e.g., oval, triangular, rectangular, star, and different sizes for different holes are contemplated. When the frangible ampoule 160 is fractured, the filter 135 prevents shards of the ampoule 160 from reaching the absorbent component 110. Thus, the filter 135 is made sufficiently thick so as not to be pierced by the shards of the frangible ampoule 160 after the frangible ampoule 160 is broken.

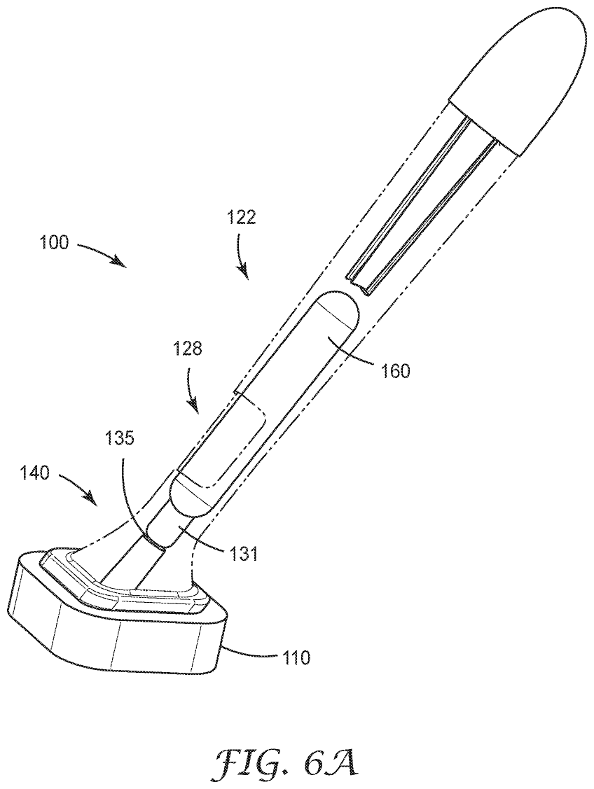

The handle 122 may optionally include one or more ribs 130 on the inner surface 126 of the body 120, as illustrated in FIGS. 5 and 6. The ribs 130 may extend longitudinally along all or part of the length of the handle from the filter 135 to the proximal portion 124 of the body. When force is applied to activation region 128, e.g., by the hand of an operator wishing to coat a surface with the solution contained in the frangible ampoule 160, the ribs 130 act as stress concentrators, thereby decreasing the amount of force required to fracture the wall 162 of the frangible ampoule 160. The shape of a rib 130 may be selected to optimize its function as a stress concentrator, and may be, for example, approximately square, rectangular, triangular, or semicircular in cross-section. In some embodiments the handle 122 may include one or more ampoule spacers 131 on the inner surface 126 of the body 120 and extending longitudinally from the filter 135 as shown in FIG. 6a. The ampoule spacers 131 are configured to support an end of the ampoule 160 to create a distance (e.g., 0.75 cm) between the ampoule 160 and the filter 135. Creating such a distance, i.e., ampoule space, between the ampoule 160 and the filter 135 may allow for unimpeded flow of contents released from the frangible ampoule 160 after activation. Creating such a distance also allows the activation region 128 to be located further from the distal end 123 of the applicator 100 while still allowing for breaking of the ampoule 160 nearer to an end of the ampoule 160, thus minimizing the chance of creating large “domes” that can retain contents of the ampoule 160 in the handle 122 after activation.

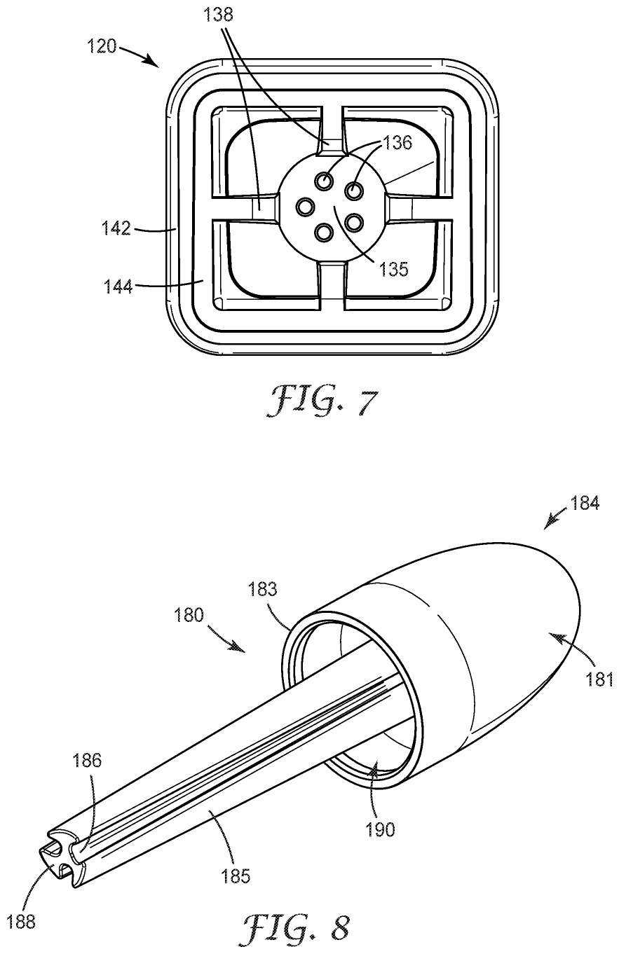

Referring now to FIG. 7, the head 140 may include one or more buttresses 138, which may extend longitudinally all or a portion of the distance from filter 135 to the distal portion 123 of the body 120. Buttresses 138 can serve, for example, to improve the rigidity of the head 140 and to direct the flow of a substance released from a fractured frangible ampoule, thereby improving the ease and efficiency with which the applicator 100 is used. The distal ends of the buttresses 138 can also be melted in the hot plate welding process previously described to provide improved attachment of the absorbent component 110. The buttresses 138 may also function to support the absorbent component 110.

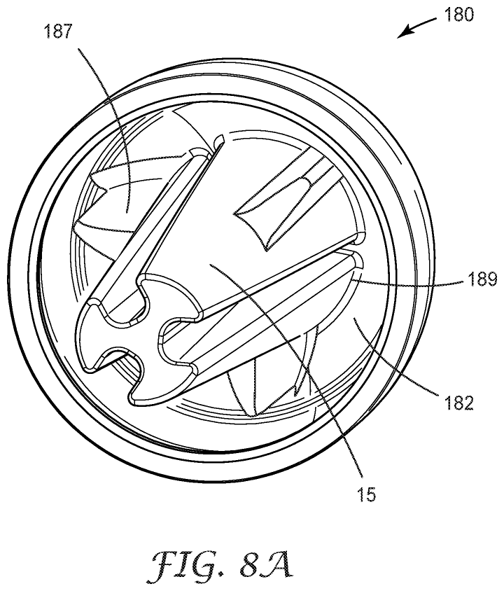

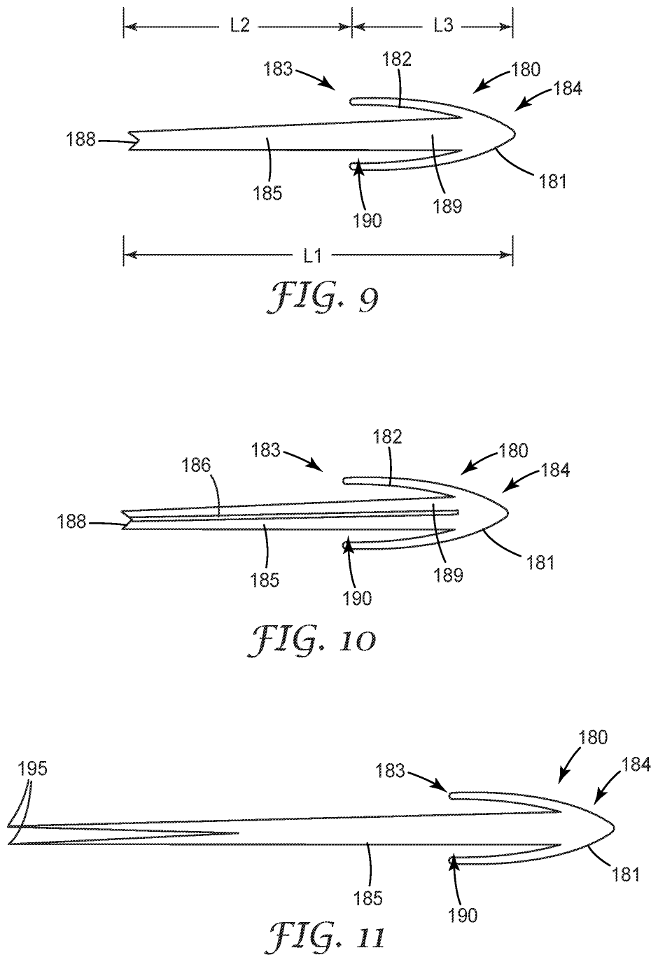

The applicator 100 further comprises cap 180 and spacer 185. As shown in FIGS. 8-13, the cap 180 has exterior surface 181, an interior surface 182, a distal portion 183, and a proximal portion 184. As shown in FIG. 9, the cap 180 and spacer 185 combine to create a longitudinal length L1, the spacer 185 extending distally beyond the distal end 183 of the cap 180 by a length L2, and the cap 180 having a longitudinal length L3 that extends longitudinally from the most proximal point of the exterior surface 181 to the point at which the exterior surface and interior surface of the cap meet. Spacer 185 has a distal end 188 and a proximal end 189, the spacer 185 projecting away from the interior surface 182 of the cap 180. As shown in FIG. 8a, in some embodiments the cap 180 and spacer 185 may include one or more fins 187, the fins 187 protruding from the proximal end 189 of spacer 185 and joined to a portion of the interior surface 182 of the cap 180.

As shown in FIGS. 8-11, the spacer 185 projects from the proximal portion 184 of the interior surface 182 of the cap 180, and the distal end 188 of the spacer 185 extends distally beyond the distal end 183 of the cap 180. In certain embodiments, the spacer 185 extends distally beyond the distal end 183 of the cap 180 by a length L2, as shown in FIG. 9. In some embodiments, the length L2 is at least 25%, at least 50%, at least 75%, at least 100%, at least 125%, or at least 150% of the length L3, i.e., the longitudinal length of the cap 180. In some embodiments, the length L2 is less than 500%, less than 400%, less than 300%, less than 200%, or less than 150% (e.g., 140% to 150%) of the length L3. In some embodiments, the length L2 is 50% to 500%, 75% to 400%, 100% to 300%, 125% to 200%, 130% to 170%, or 135% to 160% of the length L3.

In some embodiments, length L1 is at least 50%, at least 75%, at least 90%, or at least 100% of the longitudinal length of frangible ampoule 160. In some embodiments, length L1 may be equal to the longitudinal length of frangible ampoule 160. In some embodiments, length L1 is less than or equal to 800%, less than or equal to 600%, less than or equal to 400%, or less than or equal to 200% of the longitudinal length of frangible ampoule 160. In some embodiments, length L1 is 50% to 800%, 75% to 600%, 90% to 400%, or 100% to 200% (e.g., 120%) of the longitudinal length of frangible ampoule 160.

In some embodiments, length L1 may be at least 10%, at least 20%, at least 30%, at least 40%, or at least 50% of the length of the hollow body 120 as measured along the longitudinal length from filter 135 to the proximal portion 124 of the hollow body 120. In some embodiments, length L1 may be less than or equal to 120%, less than or equal to 100%, less than or equal to 90%, less than or equal to 80%, or less than or equal to 70% of the length of the hollow body 120 as measured along the longitudinal length from filter 135 to the proximal portion 124 of the hollow body 120. In some embodiments, length L1 may be 10% to 120%, 20% to 100%, 30% to 90%, 40% to 80%, or 50% to 70% (e.g., 67%) of the length of the hollow body 120 as measured along the longitudinal length from filter 135 to the proximal portion 124 of the hollow body 120.

In some embodiments and as shown in FIGS. 2, 4, 8-10, 11, and 12, the distal end 188 of the spacer 185 may have concave curvature. The concave curvature may be selected to complement the convex curvature of an end of an exterior surface of the frangible ampoule 160. That is, the radius of curvature of the exterior end surface of the frangible ampoule 160 and the radius of curvature of the distal end 188 are approximately equal. Thus, the length of spacer 185 and the curvature of the distal end 188 may be selected to maintain the frangible ampoule in a desired position, such as, for example, maintaining the frangible ampoule in the activation region 128 of the body 120. The spacer 185 may optionally include a channel 186, as shown in FIGS. 2, 8, and 10.

In other embodiments, and as shown in FIG. 11 the spacer 185 may include fingers 195. Fingers 195 may be selected to have a length and flexibility sufficient to hold the frangible ampoule 160 in place without having to complement the curvature of the end of the exterior surface of the frangible ampoule 160. Fingers 195 may be straight as shown or may be curved, such as, for example, in the shape of a helix or spring. As shown in FIG. 11, the spacer 185 may include two fingers 195, though embodiments including more than two fingers 195 are contemplated. Fingers 195 may be useful, for example, when a particular cap 180, spacer 185, and body 120 of an applicator 100 are used with frangible ampoules 160 of varying lengths, shapes, and/or curvatures. In some embodiments, the fingers 195 may be formed from the same material as the spacer 185. In some embodiments, the fingers 195 may be formed as a unitary structure with the spacer 185. In some embodiments, the fingers 195 may be constructed using a material that is less rigid, i.e., more flexible, than the material used to form the spacer 185, and may be joined to the spacer 185 by means known in the art, such as, for example, with an adhesive, a heat seal, a spin weld, an ultrasonic weld, and combinations thereof.

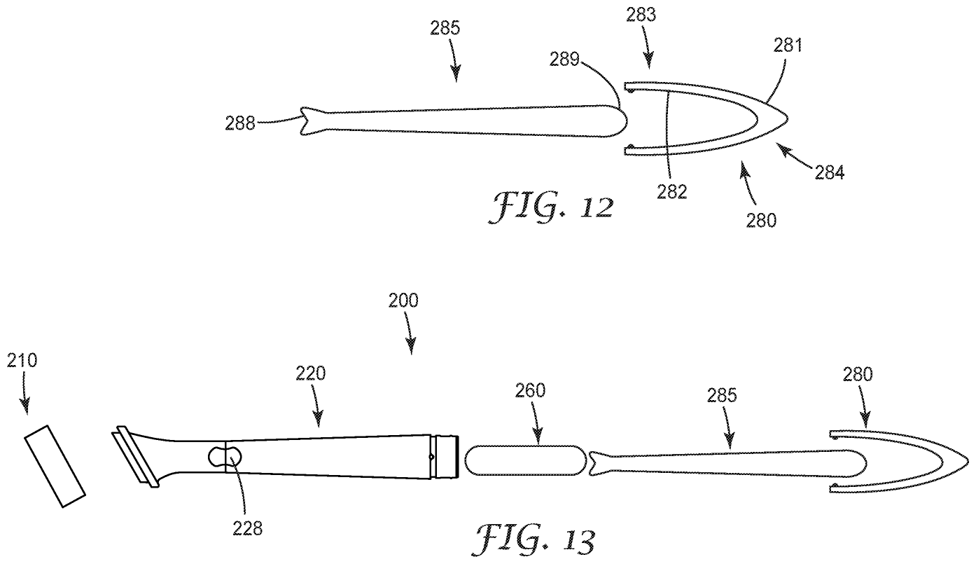

In some embodiments, the cap 180 and the spacer 185 may be formed as a unitary piece, such as by, for example, injection molding. In other embodiments, the cap 180 and the spacer 185 may be formed as separate components which could be joined by means known in the art, such as, for example, with an adhesive, a heat seal, a spin weld, an ultrasonic weld, and combinations thereof, or the cap 180 and the spacer 185 could remain as separate components in the assembled applicator 200. For example, as shown in FIGS. 12 and 13, a cap 280 has exterior surface 281, interior surface 282, distal portion 283, and proximal portion 284; and spacer 285 has distal end 288 and proximal end 289. The distal end 288 of spacer 285 may include a concave curvature, and the proximal end 289 may include a convex curvature. The concave curvature may be selected to complement the convex curvature of an end of an exterior surface of a frangible ampoule. In certain embodiments, the convex curvature of the proximal end 289 is selected to complement the concave curvature of the interior surface 282 of the cap. By selection of length of the spacer 285 and the curvature of its ends, an applicator 200 may be obtained that maintains the frangible ampoule 260 in a desired position in an applicator, i.e., in a position where at least a portion of the frangible ampoule 260 is adjacent to the activation region 228.

The cap 180 is adapted to be attached to the hollow body 120 by means of a snap fit. Referring to FIG. 8, this is accomplished by providing a retention ring 190 inwardly projecting from side walls adjacent to the major orifice of the cap 180 and which has a smaller inside diameter than the outside diameter of the outwardly projecting ridge of attachment feature 154 (FIG. 3) near the end of the proximal portion of the body. When the applicator is assembled, the retention ring 190 of the cap is snapped over the outwardly projecting ridge of attachment feature 154 on the body. Referring again to FIG. 3, a seal ring 152 may be optionally provided to prevent leakage of the contents of the applicator after the frangible ampoule is fractured.

In order to avoid creating a vacuum and restricting flow through the applicator, a means of maintaining atmospheric pressure in the device is preferably employed. One or more vents in the body functions to aspirate air into the internal volume of the applicator as the substance flows into the absorbent component to maintain atmospheric pressure within the device and prevent “air locking.” As shown in FIG. 3, vent 150 may be a hole located in the proximal portion of body 120. Preferably, the geometry and location of the vent would not result in leakage of substance from the device. To prevent an applicator from leaking, it has been found that a particularly advantageous location for a vent hole is in a location where a portion of the vent hole is covered by the distal end of the cap 180, as shown in FIG. 1.

Referring to FIG. 2, in an embodiment, an applicator 100 may be assembled by attaching an absorbent component 110 to a hollow body 120 using any of the methods previously described; placing a frangible ampoule 160 in the hollow body 120; and attaching cap 180 with integral spacer 185. Referring to FIG. 12, in another embodiment, an applicator 200 may be assembled by attaching an absorbent component 210 to a hollow body 220; placing a frangible ampoule 260 in the hollow body; placing a spacer 285 in the hollow body; and attaching cap 280.

Generally, the applicators of the present disclosure can be made by known methods. In some embodiments, injection molding may be used. A variety of materials may be used to form the applicators. In some embodiments, applicators formed of low flexural modulus materials such as low-density polyethylene may be used. For example, in some embodiments, materials having a flexural modulus of no greater than 500 Mpa, e.g., no greater than 400 MPa, or even no greater than 350 MPa may be used.

Applicator bodies 120 may be molded from polymers such as, for example, polyethylenes, linear low density polyethylenes, medium density polyethylenes, high density polyethylenes, branched polyethylenes, polypropylenes, and combinations thereof. Applicator bodies of the present disclosure were molded from a combination of linear low-density polyethylene and medium density polyethylene and used to make applicators of the present disclosure.

Applicator caps 180 and spacers 185 may be molded from polymers such as, for example, polyethylenes, linear low-density polyethylenes, medium-density polyethylenes, high-density polyethylenes, branched polyethylenes, polypropylenes, and combinations thereof. Caps including spacers, as shown in FIG. 7, may be molded from high-density polyethylene and used to make applicators 100 of the present disclosure.

### Method of Use of the Applicator

A substance, such as those described above, may be applied to a surface with an applicator as described in the present disclosure by releasing the substance from the frangible ampoule, e.g., the user holding the applicator along the palm of his/her hand and squeezing the applicator between a finger and thumb of that hand in the activation region such that the substance flows into the absorbent component when the frangible ampoule is fractured, and then applying the substance to a surface by pressing the absorbent component to the surface.

In some embodiments, the surface comprises mammalian tissue such as, for example, skin, bone, cartilage, enamel, dentin, cementum, dental pulp, gum tissue, mucous membrane, organ tissue, epithelial tissue, ocular tissue, tympanic tissue, connective tissue, muscular tissue, nervous tissue, and combinations thereof. In some embodiments, the mammalian tissue may include a perforation such as, for example, incision, an abrasion, a laceration, a puncture, an orifice, and combinations thereof.

Various modifications and alterations of the applicator 100 of the present disclosure will become apparent to those skilled in the art without departing from the scope and spirit of this disclosure.

All cited references, patents, and patent applications in the above application for letters patent are herein incorporated by reference in their entirety in a consistent manner. In the event of inconsistencies or contradictions between portions of the incorporated references and this application, the information in the preceding description shall control. The preceding description, given in order to enable one of ordinary skill in the art to practice the claimed invention, is not to be construed as limiting the scope of the disclosure, which is defined by the claims and all equivalents thereto.

1. An applicator for applying a substance to a surface, the applicator comprising:
a hollow body, wherein the hollow body includes a distal portion, a proximal portion, an exterior surface, an interior surface, an activation region, and a filter;
a frangible ampoule, wherein the frangible ampoule contains the substance;
a cap, wherein the cap includes a proximal portion, a distal portion, an exterior surface, and an interior surface, and;
a spacer, wherein the spacer includes a distal end and a proximal end,
wherein the frangible ampoule is located in the hollow body between the filter and the distal end of the spacer, wherein at least a portion of the frangible ampoule is located in the activation region, and wherein the spacer is located between the frangible ampoule and the cap.

2. The applicator of claim 1, wherein the hollow body includes a vent.

3. The applicator of claim 1, wherein the hollow body has a frustoconical geometry and tapers along the longitudinal axis of the hollow body from the proximal portion to the distal portion.

4. The applicator of claim 1, wherein the hollow body is formed of a polymer.

5. (canceled)

6. (canceled)

7. The applicator of claim 1, wherein the frangible ampoule abuts the filter.

8. The applicator of claim 1, wherein the longitudinal length of the spacer and cap L1 is at least 50% of the longitudinal length of the frangible ampoule.

9. The applicator of claim 1, wherein the spacer extends distally beyond the distal end of the cap by a length L2, wherein L2 is 50% to 500% of the longitudinal cap length L3.

10. The applicator of claim 1, wherein the longitudinal length of the spacer and cap L1 is 50% to 800% of the longitudinal length of frangible ampoule.

11. The applicator of claim 1, wherein the cap and the hollow body are joined with an adhesive, a snap fit mechanism, a threaded mechanism, a heat seal, a spin weld, an ultrasonic weld, and combinations thereof.

12. The applicator of claim 1, wherein the cap covers a portion of the vent.

13. (canceled)

14. (canceled)

15. (canceled)

16. The applicator of claim 1, wherein the spacer includes a channel.

17. (canceled)

18. The applicator of claim 1, wherein the distal end of the spacer abuts the frangible ampoule.

19. The applicator of claim 1, wherein the proximal end of the spacer has convex curvature.

20. The applicator of claim 1, wherein the proximal end of the spacer abuts the interior surface of the proximal portion of the cap.

21. (canceled)

22. (canceled)

23. The applicator of claim 1, wherein the spacer and the cap are combined.

24. (canceled)

25. (canceled)

26. The applicator of claim 1, wherein the spacer includes a helical portion.

27. The applicator of claim 1, wherein the activation region includes a rib, wherein the rib extends from the interior surface of the body.

28. The applicator of claim 1, wherein the interior surface of the body further comprises an ampoule spacer.

29. The applicator of claim 1, wherein the substance is selected from the group consisting of a liquid, a gel, a suspension, a paste, and combinations thereof.

30. (canceled)

31. (canceled)

32. The applicator of claim 1, wherein the applicator further comprises an absorbent component.
